(19) United States
(12) Patent Application Publication
(10) Pub. No.: US 2011/0301401 A1
(43) Pub. Date: Dec. 8, 2011

(54) COMPOSITIONS AND METHODS FOR THERMORADIOTHERAPY

(76) Inventors: Andrew C. Larson, Kildeer, IL (US); Andrew C. Gordon, Chicago, IL (US); Reed A. Omary, Wilmette, IL (US)

(21) Appl. No.: 13/155,925

(22) Filed: Jun. 8, 2011

Related U.S. Application Data
(60) Provisional application No. 61/352,625, filed on Jun. 8, 2010.
(60) Provisional application No. 61/374,455, filed on Aug. 17, 2010.

(51) Int. Cl. A61N 5/02; A61P 35/00; A61K 51/02

(52) U.S. Cl. 600/2; 424/1.61

(57) ABSTRACT

The present invention relates to compositions and methods for increasing tissue-radiosensitivity through induction of local hyperthermia. In particular, the present invention provides superparamagnetic, paramagnetic, or ferromagnetic radioactive particles that heat surrounding tissue upon magnetic induction, and methods of use thereof. In some embodiments, the present invention provides compositions and methods for thermoradiotherapy (e.g. anti-tumor therapy).

The present application claims priority to U.S. Provisional Application Ser. No. 61/352,625 filed Jun. 8, 2010, and U.S. Provisional Application Ser. No. 61/374,455 filed Aug. 17, 2010, which are herein incorporated by reference in their entireties.

### FIELD OF THE INVENTION

The present invention relates to compositions and methods for increasing tissue-radiosensitivity through induction of local hyperthermia. In particular, the present invention provides superparamagnetic, paramagnetic, or ferromagnetic radioactive particles that heat surrounding tissue upon magnetic induction, and methods of use thereof. In some embodiments, the present invention provides compositions and methods for thermoradiotherapy (e.g. anti-tumor therapy).

### BACKGROUND

Multiple loco-regional oncologic therapies involve injection and/or implantation of radioactive materials with the purpose of providing locally high doses of activity to malignant tissues while minimizing radiation exposure to normal surrounding tissues.

### SUMMARY OF THE INVENTION

In some embodiments, the present invention provides incorporation of magnetic materials into particles comprising one or more radioactive elements (e.g. yttrium, holmium, etc.) to permit heating of local tissue to enhance radiosensitivity. In some embodiments, a radioactive particle (e.g. comprising yttrium, holmium, etc.) is coated or externally labeled with magnetic materials (e.g. iron-oxide containing materials). In some embodiments, magnetic materials are incorporated into the internal composition of radioactive particles. In one particular embodiment, a magnetic material (e.g. iron oxide) is directly incorporated into radioactive particles (e.g. Yttrium-90 THERASPHERES).

In some embodiments, the present invention provides administration of superparamagnetic, paramagnetic, or ferromagnetic radioactive particles to a tissue of interest (e.g. within a subject, a tumor, etc.). In some embodiments, a magnetic radioactive particle is delivered to a tissue of interest (e.g., malignant tumor), followed by local hyperthermia treatments. In some embodiments, local hyperthermia treatment comprises positioning the tissue of interest within a rapidly switching magnetic field. In some embodiments, the rapidly switching magnetic field causes the superparamagnetic, paramagnetic, or ferromagnetic radioactive particles to heat their local environment (e.g. local tissue, surrounding tumor, etc.). In some embodiments, heating of tissue surrounding a superparamagnetic, paramagnetic, or ferromagnetic radioactive particle enhances the susceptibility of the tissue to radioactive treatment. In some embodiments, heating surrounding tissue enhances the effectiveness of the radioactivity in killing cells in the surrounding tissue (e.g. tumor cells). In some embodiments, the heating is locally focused, thereby only increasing radioactive sensitivity in and/or near the tissue of interest.

In some embodiments, compositions and methods of the present invention permit use of lower overall radiation doses when compared to conventional oncologic radioactivity therapies. In some embodiments, compositions and methods of the present invention improve loco-regional tissue selectivity by directing magnetization toward the tissue of interest.

In some embodiments, the present invention provides a method of killing tissue in a subject comprising: (a) delivering superparamagnetic, paramagnetic, or ferromagnetic radioactive microparticles to undesired tissue in said subject; and (b) applying alternating electromagnetic field to said undesired tissue. In some embodiments, the subject suffers from cancer. In some embodiments, the undesired tissue comprises tumor cells. In some embodiments, the superparamagnetic, paramagnetic, or ferromagnetic radioactive microparticles comprise iron oxide. In some embodiments, the superparamagnetic, paramagnetic, or ferromagnetic radioactive microparticles comprise yttrium and/or holmium. In some embodiments, the superparamagnetic, paramagnetic, or ferromagnetic radioactive microparticles comprise yttrium oxide-aluminosilicate glass. In some embodiments, the superparamagnetic, paramagnetic, or ferromagnetic radioactive microparticles comprise yttrium-90 microparticles.

In some embodiments, the present invention provides a microparticle composition comprising a therapeutic radioactive material, and a superparamagnetic, paramagnetic, or ferromagnetic material. In some embodiments, the therapeutic radioactive material is configured for therapeutic delivery of radiation from said microparticle. In some embodiments, the radioactive material comprises yttrium and/or holmium. In some embodiments, the superparamagnetic, paramagnetic, or ferromagnetic material comprises iron oxide. In some embodiments, the microparticle comprises aluminosilicate glass.

### DESCRIPTION OF THE DRAWINGS

FIG. 1 shows a plot of thermal response as a function of treatment time with various microparticles.

FIG. 2 shows a plot of thermal response as a function of treatment time with various microparticles.

### DEFINITIONS

As used herein, the term “microparticle” refers to any spherical or substantially spherical particles as well as particles that are not necessarily spherically shaped with diameter in the micrometer range, typically 1 μm to 1000 μm (e.g., 1 μm . . . 2 μm . . . 5 μm . . . 10 μm . . . 20 μm . . . 50 μm . . . 100 μm . . . 200 μm . . . 500 μm . . . 1000 μm), although “microparticles” may be up to 5 mm in diameter. The terms “microparticle” and “particle” are inclusive of any spherical or substantially spherical particles termed “microspheres.” “Microparticles” may comprise various natural and synthetic materials including, but not limited to glass, polymers, and ceramics. “Microparticles” may be solid, hollow, porous, or combinations thereof.

### DETAILED DESCRIPTION

The present invention relates to compositions and methods for increasing tissue-radiosensitivity through induction of local hyperthermia. In particular, the present invention provides superparamagnetic, paramagnetic, or ferromagnetic radioactive particles that heat surrounding tissue upon magnetic induction, and methods of use thereof. In some embodiments, the present invention provides compositions and methods for thermoradiotherapy (e.g. anti-tumor therapy). In some embodiments, the present invention provides compositions and methods for therapy (e.g. cancer therapy) via both hyperthermia and radiotherapy (e.g. selective internal radiation therapy (SIRT)). In some embodiments, compositions and methods provide thermoradiotherapy. In some embodiments, the present invention provides hyperthermia induction and radiotherapy functionalities in a single particle. In some embodiments, the present invention provides hyperthermia induction, radiotherapy, and magnetic resonance mapping functionalities in a single particle.

In some embodiments, the present invention provides particles and/or microparticles to deliver radioactivity and hyperthermic-induction functionality to a tissue (e.g. within a subject). In some embodiments, microparticles further deliver one or more therapeutics, imaging functionality, and/or other functionality to a tissue within a subject. In some embodiments, the present invention provides microparticles (e.g. glass microparticles) comprising one or more radioisotopes (e.g. for use in radiotherapy). Exemplary glass microparticles are described in U.S. Pat. No. 4,789,501; U.S. Pat. No. 5,011,677; and U.S. Pat. No. 5,302,369; although the microparticles of the present invention are not limited to those described therein.

In some embodiments, microparticles of the present invention comprise one or more radioisotopes (e.g. yttrium, holmium, cobalt, etc.). In some embodiments, radioisotopes (e.g. yttrium, holmium, cobalt, etc.) are incorporated into, distributed throughout, or attached to the surface of microparticles. In some embodiments, microparticles comprise yttrium oxide-aluminosilicate glass (e.g. yttrium-90 microparticles). In some embodiments, microparticles comprise yttrium aluminosilicate. In some embodiments, microparticles comprise holmium oxide-aluminosilicate glass. In some embodiments, microparticles comprise radioactive material. In some embodiments, radioactive materials are not limited to metaiodobenzylguanidine (MIBG), iodine-131, lutetium-177, yttrium-90, strontium-89, and samarium-153, lexidronam radioactive holmium, cobalt-60, etc. In some embodiments, any compounds or materials that may find use in radioisotope therapy are suitable for the use with the present invention.

In some embodiments, microparticles of the present invention are between 1 and 5000 μm in diameter (e.g. 1 μm . . . 2 μm . . . 5 μm . . . 10 μm . . . 20 μm . . . 50 μm . . . 100 μm . . . 200 μm . . . 500 μm . . . 1 mm . . . 2 mm . . . 5 mm). In some embodiments, microparticles of the present invention are between 10 and 100 μm in diameter (e.g. 10 μm . . . 20 μm . . . 30 μm . . . 40 μm . . . 50 μm . . . 60 μm . . . 70 μm . . . 80 μm . . . 90 μm . . . 100 μm).

In some embodiments, microparticles of the present invention comprise one or more magnetic, paramagnetic, and/or superparamagnetic, paramagnetic, or ferromagnetic compounds (e.g. iron oxide, magnetite, etc.). In some embodiments, superparamagnetic, paramagnetic, or ferromagnetic compounds are incorporated into, distributed throughout, or attached to the surface of microparticles. In some embodiments, superparamagnetic, paramagnetic, or ferromagnetic compounds are a constituent of microparticles. In some embodiments, microparticles comprise one or more iron oxides (e.g. Fe₃O₄). In some embodiments, microparticles comprise magnetite. In some embodiments, suitable magnetic compounds (e.g., superparamagnetic, paramagnetic, or ferromagnetic compounds) for use in the present invention include, but are not limited to iron oxides, MnSO₄, FeSO₄, CoCl₂, NiSO₄, ZnSO₄, K₄Fe(CN)₆, [Co(NH3)₆]Cl₃, [Ni(NH₃)6]Cl₂, etc.

In some embodiments, the present invention provides delivery of microparticles comprising radioisotopes and superparamagnetic, paramagnetic, or ferromagnetic compounds. In some embodiments, superparamagnetic, paramagnetic, or ferromagnetic radioactive microparticles are delivered to selected tissue (e.g. tumor tissue). In some embodiments, superparamagnetic, paramagnetic, or ferromagnetic radioactive microparticles are concentrated in a selected tissue (e.g. tumor). In some embodiments, superparamagnetic, paramagnetic, or ferromagnetic radioactive microparticles kill surrounding cells and tissue (e.g. tumors) via radiotherapy. In some embodiments, superparamagnetic, paramagnetic, or ferromagnetic compounds (e.g. iron oxide) provide an imaging functionality to compositions of the present invention. In some embodiments, microparticles comprising superparamagnetic, paramagnetic, or ferromagnetic compounds (e.g. iron oxide) are imagable within a tissue or subject by a variety of imaging methods including magnetic resonance imaging (MRI). In some embodiments, superparamagnetic, paramagnetic, or ferromagnetic compounds (e.g. iron oxide) provide heat-induction functionality to compositions of the present invention. In some embodiments microparticles comprising superparamagnetic, paramagnetic, or ferromagnetic compounds (e.g. iron oxide) are heat-inducible by exposure to an alternating magnetic field.

In some embodiments, the present invention provides compositions and methods to increase the sensitivity (e.g. radiosensitivity) of surrounding tissue (e.g. tumors) to radiotherapy. In some embodiments, the present invention provides magnetic induction heating procedures to heat tissues (e.g. tumor tissue) local to the position of radioactive therapeutic particles. In some embodiments, radioactive particles (e.g. yttrium-containing particles, holmium-containing particles, superparamagnetic, paramagnetic, or ferromagnetic radioactive particles, etc.) comprise ferromagnetic materials (e.g. via labeling, exterior coating, or as part of the internal composition of the radioactive particle) to permit magnetic inductive heating of those tissues near the radioactive particle. In some embodiments, compositions of the present invention heat surrounding environment (e.g., cells, tissues, tumor, etc.) upon application of electromagnetism and/or magnetic field to the composition. In some embodiments, magnetic inductive heating is achieved using a coil (e.g. positioned near tissues of interest containing the radioactive particle) and a radiofrequency electrical power source. In some embodiments, alternating current through the coil generates an alternating magnetic field. In some embodiments, an alternative magnetic field is provided in the proximity of the superparamagnetic, paramagnetic, or ferromagnetic radioactive particles in selected tissue. In some embodiments, superparamagnetic, paramagnetic, or ferromagnetic radioactive particles heat the surrounding tissue when exposed to alternating magnetic current. In some embodiments, heating of the local tissue (e.g. tumor) causes hyperthermia. In some embodiments, hyperthermia sensitizes the local tissue (e.g. tumor) to radiation, thereby resulting in increased localized cell death. In some embodiments, heat inducing methods of the present invention increase the temperature of the surrounding tissue by at least 1° C. (e.g. 2° C. . . . 3° C. . . . 4° C. . . . 5° C. . . . 10° C. . . . 20° C. . . . 50° C., etc.). In some embodiments, hyperthermia increases tissue sensitivity to radiotherapy by at least 10% (e.g. about 10% . . . about 20% . . . about 30% . . . about 50% . . . about 75% . . . about 100% . . . about 150% . . . about 200% . . . about 500%, etc.).

In some embodiments, the present invention provides compositions and methods for causing cell death in surrounding tissue (e.g. tumor tissue). In some embodiments, superparamagnetic, paramagnetic, or ferromagnetic radioactive particles are delivered or administered to a subject (subject suffering from cancer) or tissue (e.g. tumor) via any suitable means (e.g. intravenous, direct injection, catheter delivery, systemic delivery, etc.). In some embodiments, superparamagnetic, paramagnetic, or ferromagnetic radioactive particles are delivered to a localized area (e.g. tumor). In some embodiments, superparamagnetic, paramagnetic, or ferromagnetic radioactive particles concentrate within selected tissue (e.g. tumor). In some embodiments, location of superparamagnetic, paramagnetic, or ferromagnetic radioactive particles is monitored by one or more imaging techniques (e.g. MRI). In some embodiments, alternating magnetism is applied to the region of tissue containing the superparamagnetic, paramagnetic, or ferromagnetic radioactive particles. In some embodiments, exposure of superparamagnetic, paramagnetic, or ferromagnetic radioactive particles to an alternating magnetic field induces hyperthermia in the surrounding tissue (e.g. tumor). In some embodiments, hyperthermia of tissue sensitizes the tissue to radiation therapy. In some embodiments, the radioisotopes (e.g. yttrium) in the superparamagnetic, paramagnetic, or ferromagnetic radioactive particles cause cell death in the surrounding tissue (e.g. heat-sensitized tissue) via radiotherapy.

In some embodiments, the present invention provides compositions and method for the treatment of cancer. In some embodiments, microparticles of the present invention cause cell death in surrounding cancer (e.g. tumor) cells when exposed to electromagnetic radiation. The present invention is not limited to any type of cancer or tumor. In some embodiments, the present invention provides elimination or reduction in mass of solid tumors and/or masses. In some embodiments, compositions and method of the present invention find use in treatment and/or elimination of any suitable type of solid tumor or undesired mass. In some embodiments, the present invention finds use with other cancer treatment known to those of skill in the art (e.g. chemotherapy, radiation, etc.).

### EXAMPLES

The following Examples are presented in order to provide certain exemplary embodiments of the present invention and are not intended to limit the scope thereof.

### Example 1

Yttrium aluminosilicate (YAS) microparticles (20-40 μm in diameter) were labeled with magnetite (50% by mass) and exposed to various heat treatments: no heat, 750° C. for varied time periods in a reducing or oxidizing atmosphere (Table 1). A control sample of 2 mL H₂O was used. The heating properties of these various particles were compared to those of pure magnetite beads (also 20-40 μm in diameter) for equivalent field strengths and masses of Fe₃O₄ in 2 mL H₂O. This allowed for identification of the most effective treatment process to produce magnetic YAS microparticles specifically designed for hyperthermic applications.

TABLE 1

| | Long | Long | Long | Long | Short | | | |
|---|---|---|---|---|---|---|---|---|
| | Oxidizing | Reducing | Reducing | Reducing | Reducing | No | Magnetite | Water |
| | Treatment | Treatment | Treatment | Treatment | Treatment | Treatment | Microspheres | Control |
| Samples | (72.5 mg/mL | (72.5 mg/mL | (36.25 mg/mL | (108.75 mg/mL | (72.5 mg/mL | (72.5 mg/mL | (72.5 mg/mL | (0 mg/mL |
| (particle conc.) | Fe₃O₄) | Fe₃O₄) | Fe₃O₄) | Fe₃O₄) | Fe₃O₄) | Fe₃O₄) | Fe₃O₄) | Fe₃O₄) |
| Total Mass of | 145 mg | 145 mg | 72.5 mg | 217.5 mg | 145 mg | 145 mg | 145 mg | 0 mg |
| Sample Labeled | | | | | | | | |
| with Fe₃O₄ | | | | | | | | |
| Total Mass of | 290 mg | 290 mg | 145 mg | 435 mg | 290 mg | 290 mg | 0 mg | 0 mg |
| YAS Particle | | | | | | | | |
| Sample | | | | | | | | |
| Particle | 20-40 μm | 20-40 μm | 20-40 μm | 20-40 μm | 20-40 μm | 20-40 μm | 20-40 μm | N/A |
| Diameter | | | | | | | | |
| Percent Labeled | 50% | 50% | 50% | 50% | 50% | 50% | 100% | 0% |
| with Fe₃O₄ (by | | | | | | | | |
| mass) | | | | | | | | |
| Volume of H₂O | 2 mL | 2 mL | 2 mL | 2 mL | 2 mL | 2 mL | 2 mL | 2 mL |
| Treatment | 750° C. | 750° C. | 750° C. | 750° C. | 750° C. | N/A | N/A | N/A |
| Temperature | | | | | | | | |
| Time of | 4 hours | 4 hours | 4 hours | 4 hours | 1 hour | 0 hours | N/A | N/A |
| Treatment | | | | | | | | |
| Graphite | − | + | + | + | + | − | N/A | N/A |
| Crucible | | | | | | | | |
| Dense Al₂O₃ | + | − | − | − | − | − | N/A | N/A |
| Crucible | | | | | | | | |

Samples were placed in a water-cooled Helmholtz-style coil consisting of two 2-turn windings separated by 2.25 inches, each winding with an inner diameter of 2.5 inches and a length of 1 inch. The fiber optic temperature probe was attached (via a converter) to a PC enabled for data acquisition. Baseline temperatures were recorded for 1 minute after which time the alternating magnetic field (AMF) was applied for 24 minutes. Temperature continued to be recorded after removal of the AMF.

The AMF was produced via a 2.4 kW radiofrequency (RF) generator yielding a controlled current through the coil (specified by the operator) alternating at a specific frequency in the 150-400 kHz range. This RF generator has been thoroughly tested and certified to produce consistent, reproducible currents through the specified coil. Current through the coil was chosen based on the desired field strength. The electromagnetic radiation involved in this example is non-ionizing. Furthermore, the frequency range involved in this procedure is the same frequency range used for AM radio broadcasting.

It should be noted that AMFs can induce eddy currents in normal tissues mildly increasing the temperature in those tissues. RF induction was chosen for this application because it can penetrate tissues, such as subcutaneous fat, without excessive heating. The induced electric fields will be parallel to the tissue interface meaning that if any heating results from eddy currents, this heating will reside in muscle rather than fat. The pattern of heating generated by the inductive applicator is toroidal in shape with a null at the center of the coil. Therefore, internal organs are much less likely to be slightly heated via eddy currents than peripheral tissues. In studies by Atikinson et al [4] and Stauffer et al [5] it has been determined that to minimize these eddy currents the product of the field strength (H) and the frequency (f) should satisfy:

H·f<4.85×10⁸ A/m.sec

if the diameter of the exposed tissue is 30 cm. A study by Brezovich [6] determined that the deposited power is proportional to this diameter squared. Therefore, assuming that the diameter of the exposed tissue is 3.8 cm, at 200 kHz the maximum field strength should be 15 kA/m and at 400 kHz the maximum field strength should be 7.5 kA/m. The AMF was used to transfer electromagnetic energy to the magnetic 90Y microparticles, heating the microparticles that received the appropriate treatment.

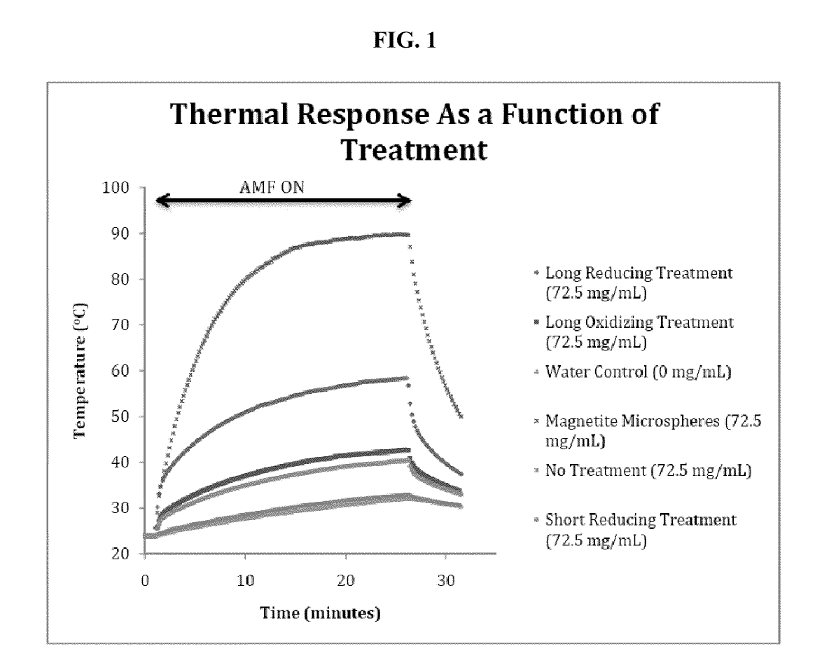

Magnetic YAS microparticles allowed heating of samples to therapeutic temperatures (above 43° C.) in as little as 5 minutes of AMF exposure (SEE FIG. 1). For a given field strength and duration of AMF exposure, the microparticles heated for 4 hours at 750° C. in a reducing atmosphere demonstrated the greatest increase in temperature. In contrast, microparticles that received no heat treatment behaved similarly to the control upon AMF exposure despite the fact that they were labeled with Fe₃O₄. Therefore, labeling with Fe₃O₄ was not a sufficient condition for achieving therapeutic temperatures upon AMF exposure.

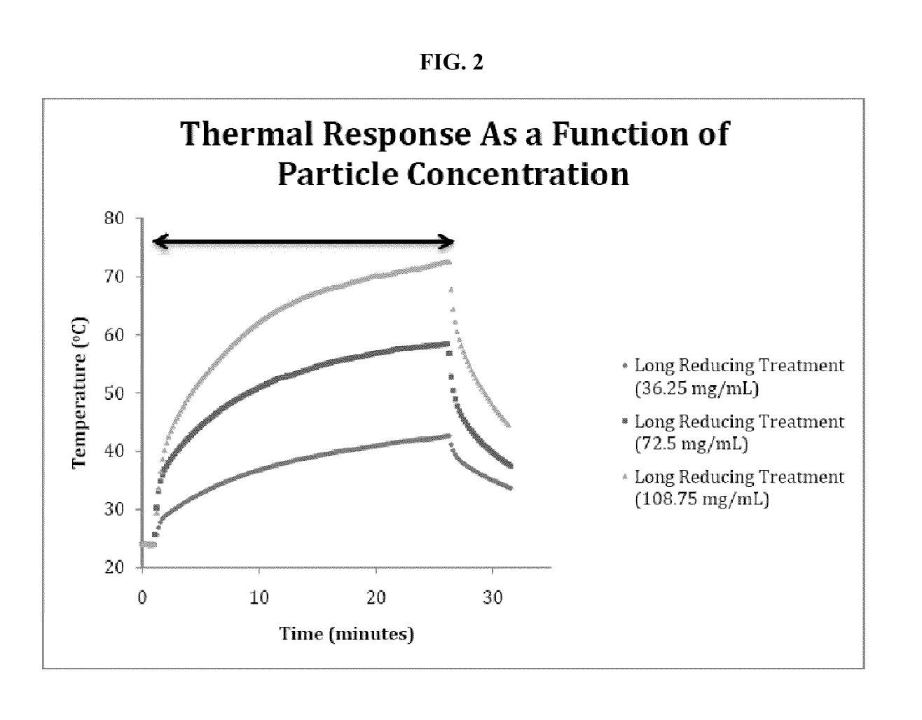

The YAS microparticles magnetically labeled by the long reducing treatment were then selected for further studies (SEE FIG. 2). Three samples of these YAS microparticles were prepared giving concentrations of Fe₃O₄: 36.25 mg/mL, 72.5 mg/mL, and 108.75 mg/mL. This allowed for determination of the relationship between microparticles concentration and thermal response to a given AMF. All samples were exposed to the same field strength. It was observed that thermal response is proportional to microparticles concentration for a given field strength and concentration of magnetite/sphere. These studies demonstrate that magnetic YAS microparticles can be used to achieve therapeutic temperatures upon exposure to an AMF.

### REFERENCES

Horsman, M. R. and J. Overgaard (2007). “Hyperthermia: a potent enhancer of radiotherapy.” Clinical Oncology (Royal College of Radiologists) 19(6): 418-26.
Gupta, T., S. Virmani, et al. (2008). “MR tracking of iron-labeled glass radioembolization microspheres during transcatheter delivery to rabbit VX2 liver tumors: feasibility study.” Radiology 249(3): 845-54.
Hafeli, U. O., S. M. Sweeney, et al. (1995). “Effective targeting of magnetic radioactive 90Y-microspheres to tumor cells by an externally applied magnetic field. Preliminary in vitro and in vivo results.” Nuclear Medicine and Biology 22(2): 147-55.
Atkinson W A, Brezovich I A, Chakraborty D P. Usable frequencies in hyperthermia with thermal seeds. IEEE Trans Biomed Eng 1984; 31:70-75.
Stauffer P R, Cetas T C, Flechter A M, et al. Observations on the use of ferromagnetic implants for inducing hyperthermia. IEEE Trans Biomed Eng 1984; 31:76-90.
Brezovich I A. Low frequency hyperthermia: capacitive and ferromagnetic thermoseed methods. In: Palival P R, Hetzel F W, eds. Medical Physics Monograph. New York, N.Y.: American Institute of Physics, 1988; 16:82-111.
US Patent Application 20070168001—Remotely RF powered conformable thermal applicators. Xiang et al.
U.S. Pat. No. 4,735,796—Ferromagnetic, diamagnetic, or paramagnetic particles useful in diagnosis and treatment of disease. Gordon, Robert T.
U.S. Pat. No. 4,569,836—Cancer treatment by intracellular hyperthermia. Gordon, Robert T.
U.S. Pat. No. 4,303,639—Cancer treatment. Gordon, Robert T.

All publications and patents mentioned in the present application and/or listed above are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of killing tissue in a subject comprising:
a) delivering superparamagnetic, paramagnetic, or ferromagnetic radioactive microparticles to undesired tissue in said subject; and
b) applying alternating electromagnetic field to said undesired tissue.

2. The method of claim 1, wherein said subject suffers from cancer.

3. The method of claim 2, wherein said undesired tissue comprises tumor cells.

4. The method of claim 1, wherein said superparamagnetic, paramagnetic, or ferromagnetic radioactive microparticles comprise iron oxide.

5. The method of claim 1, wherein said superparamagnetic, paramagnetic, or ferromagnetic radioactive microparticles comprise yttrium and/or holmium.

6. The method of claim 1, wherein said superparamagnetic, paramagnetic, or ferromagnetic radioactive microparticles comprise yttrium oxide-aluminosilicate glass.

7. The method of claim 6, wherein said superparamagnetic, paramagnetic, or ferromagnetic radioactive microparticles comprise yttrium-90 microparticles.

8. A microparticle composition comprising:
a) a therapeutic radioactive material; and
b) superparamagnetic, paramagnetic, or ferromagnetic material.

9. The microparticle composition of claim 8, wherein said therapeutic radioactive material is configured for therapeutic delivery of radiation from said microparticle.

10. The microparticle composition of claim 9, wherein said radioactive material comprises yttrium and/or holmium.

11. The microparticle composition of claim 8, wherein said superparamagnetic, paramagnetic, or ferromagnetic material comprises iron oxide.

12. The microparticle composition of claim 8, wherein said microparticle comprises aluminosilicate glass.
